(19) United States
(12) Patent Application Publication
(10) Pub. No.: US 2020/0222531 A1
(43) Pub. Date: Jul. 16, 2020

(54) METHODS OF POPULATING PRECIPITATED PARTICLES OF A MODIFIED OR SYNTHESIZED SUBSTANCE IN A TISSUE

(71) Applicants: Robert E. Coifman, Millville, NJ (US); Catherine F. Yang, Cherry Hill, NJ (US)

(72) Inventors: Robert E. Coifman, Millville, NJ (US); Catherine F. Yang, Cherry Hill, NJ (US)

(21) Appl. No.: 16/664,849

(22) Filed: Oct. 26, 2019

Related U.S. Application Data
(63) Continuation-in-part of application No. 14/567,500, filed on Dec. 11, 2014, now abandoned.
Application No. 14/567,500 is a continuation-in-part of application No. 13/008,945, filed on Jan. 19, 2011, now Pat. No. 9,107,901.
(60) Provisional application No. 61/914,432, filed on Dec. 11, 2013.
(60) Provisional application No. 61/398,170, filed on Jun. 22, 2010.
(60) Provisional application No. 61/336,799, filed on Jan. 27, 2010.
(60) Provisional application No. 61/336,205, filed on Jan. 19, 2010.

(51) Int. Cl. A61K 39/35; A61K 39/00; A61K 9/00; A61K 47/02

(52) U.S. Cl. CPC A61K 39/35; A61K 47/02; A61K 9/0019; A61K 39/0011

(57) ABSTRACT

Provided herein are methods of populating a target tissue of interest of a subject with precipitated particles of a modified or synthesized substance of interest, in which tissue the precipitated particles remain where they are formed for a sufficient time to begin their interaction with the physiology of the recipient in the physical state and locations in which they were formed, when the unmodified/unsynthesized form of the substance does not innately have the requisite solubility properties to be delivered to such tissues within the body of a designated target tissue by precipitation. The present methods include precipitating particles of the modified or synthesized substance within the target tissue by administering a solution to the target tissue of interest of a subject. The solution includes a pharmaceutically acceptable, water-miscible, non-aqueous solvent that is freely miscible with tissue fluids of the target tissue; and the modified or synthesized substance of interest, which is modified or synthesized to confer the presently required solubility properties of being soluble in the solvent and insoluble in tissue fluids of the target tissue of interest.

### RELATED APPLICATIONS

This application is a Continuation-in-Part of U.S. application Ser. No. 14/567,500 filed on Dec. 11, 2014, which is a Continuation-in-Part of U.S. application Ser. No. 13/008,945 filed on Jan. 19, 2011, which claims the benefit of U.S. Provisional Application No. 61/336,205 filed on Jan. 19, 2010, U.S. Provisional Application No. 61/336,799 filed on Jan. 27, 2010, and U.S. Provisional Application No. 61/398,170 filed on Jun. 22, 2010, to which the present application also claims the benefit. This application also claims the benefit of U.S. Provisional Application No. 61/914,432 filed on Dec. 11, 2013. The contents of all of these applications is hereby incorporated herein in their entireties.

### FIELD

The present disclosure relates generally to methods of populating a target tissue of interest of a subject with precipitated particles of a substance that did not innately have the requisite solubility properties for precipitation by solvent dilution but which substance has been modified or synthesized to give it solubility properties that will result in precipitation of particles. The present methods include delivering or administering a solution to a target tissue of interest of a subject, which solution includes the modified or synthesized substance in a suitable solvent to a tissue of the recipient/subject, such that particles of the substance precipitate and are delivered to the target tissue of interest of the recipient, which precipitated particles remain in the target tissue. Also included herein are the solutions themselves; kits that include the solution or components thereof and optionally a device to aid in the delivery of the solution (such as a syringe) and/or instructions for delivery; and methods of making such solutions.

### BACKGROUND

Allergic contact dermatitis to the urushiols of poison ivy and poison oak is a common condition for which 100 years of prior art failed to find a way to predictably and measurably induce durable immunological tolerance in previously sensitized individuals.

Previously licensed poison ivy allergy vaccines consisted of ether extracts of fresh or dried leaves evaporated to dryness and redissolved in sterile vegetable oils for either oral dosing or subcutaneous injection. They were withdrawn from the U. S. market in 1994 for failure to demonstrate statistical efficacy.

### SUMMARY

The present inventors achieved this objective of predictably and measurably inducing immunological tolerance in previously sensitized individuals, with allergy vaccines containing concentrated solutions of the offending urushiols in ethanol, of which small volumes were injected into muscle where they would quickly lose solubility and precipitate into hundreds of millions of particles falling in the 0.5 to 5 micron size range for efficient uptake by antigen-processing cells, as the injected ethanol was diluted by the water content of available tissue fluid.

The present invention encompasses methods to similarly precipitate substances-of-interest (not just urushiol) in recipient tissues-of-interest when the substance-of-interest does not innately have the necessary solubility properties of being insoluble in water but sufficiently soluble in a pharmaceutically acceptable water-miscible solvent to allow unit doses to be similarly precipitated in target tissues by solvent dilution. The inventors named their previously reported process “Vaccine Delivery by Precipitation” (VDBP). The present invention adapts the same technology to a much broader range of not only allergy vaccines but also substances-of-interest in general and might be called “Substance Delivery by Precipitation” (SDBP).

Provided herein are methods of populating a target tissue of interest of a subject with particles of a modified or synthesized substance of interest, by precipitation, when the unmodified/unsynthesized form of the substance does not innately have the requisite solubility properties be distributed within the body of a designated target tissue by precipitation to be delivered to such tissues. The present methods include precipitating particles of the modified or synthesized substance within the target tissue by administering a solution to the target tissue of interest of a subject. The solution includes a pharmaceutically acceptable, water-miscible, non-aqueous solvent that is freely miscible with tissue fluids of the target tissue; and the modified or synthesized substance of interest, which is modified or synthesized to confer the presently required solubility properties of being soluble in the solvent and insoluble in tissue fluids of the target tissue of interest. According to example embodiments, the tissues are non-liquid tissues. According to example embodiments, the solution is a homogenous solution.

Solutions of appropriately modified candidate substances may be administered in volumes of pharmaceutically acceptable solvents that will be diluted by the water content of target tissues at rates resulting in precipitation of an effective mass, number and size distribution of particles of each such substance of interest to accomplish a purpose for which that substance is administered. According to example embodiments, the precipitation occurs immediately upon administration of the particles to the target tissue of interest.

According to non-limiting examples of the present invention, a substance of interest in a water-miscible solvent may be administered to a subject (such as a mammal or other animal) intramuscularly, transdermally or by other methods that deliver the substance of interest to a desired tissue. According to non-limiting example embodiments, a substance of interest can be an allergen given to induce tolerance in allergic or autoimmune disease, it can be a vaccine given to stimulate a protective immune response against an infectious disease or against a patient's cancer or it can be any other medicinal or therapeutic substance for which administration by a method of this invention facilitates the achievement of a therapeutic goal.

According to further non-limiting examples of the invention, a method is provided of populating a target tissue of a subject with precipitated particles of a modified or synthesized substance of interest, which precipitated particles remain in the target tissue where formed. These methods include precipitating within the target tissue particles of the modified or synthesized substance of interest, wherein the substance of interest is a substance that does not, as it exists in nature in its unmodified and un-synthesized form, have solubility properties of being insoluble in water but sufficiently soluble in a pharmaceutically acceptable water-miscible, non-aqueous solvent, such that desired doses can be administered to chosen target tissues in small enough volumes of the solvent to be diluted rapidly enough by the water content of the chosen target tissue to precipitate in particles spanning an effective size range to achieve a desired physiological or pharmacological effect. According to example embodiments, the modified or synthesized substance of interest may be modified or synthesized to confer the requisite solubility properties by at least one of the following methods: 1) selecting methods least likely to adversely affect desired functions from among standard methods of physical and/or chemical modification that will render such substances insoluble in water while doses intended for administration will remain or become soluble in small enough volumes of a pharmaceutically acceptable solvent to precipitate in an effective range of particle size, or 2) synthesizing such a substance of interest with a molecular structure that does not predominate in nature but is specifically designed or selected to give it the above solubility properties without losing the biological function the user wants to preserve.

According to example embodiments, the precipitating takes place upon administering a solution of the modified or synthesized substance of interest to the subject. The solution includes a pharmaceutically acceptable solvent that is freely miscible with the tissue fluids of the target tissue of interest; and the modified or synthesized substance of interest that is soluble in the pharmaceutically acceptable, water-miscible solvent but insoluble in tissue fluids of the target tissue of interest. In example embodiments, the solution is administered to the tissue in a volume of the pharmaceutically acceptable solvent such that the solution will be diluted by tissue fluids in the non-liquid target tissue of interest following administration, at a rate that results in precipitation of an effective mass of the modified or synthesized substance of interest with a distribution of particle sizes that will accomplish a purpose for which the substance is administered. According to example embodiments, the tissues are non-liquid tissues.

The present application further relates to methods of making the solutions disclosed herein, the solutions themselves, and kits including the solutions.

### BRIEF DESCRIPTION OF THE DRAWINGS

Illustrative embodiments of the present disclosure are herein described, by way of non-limiting example, with reference to the following accompanying Figures:

FIG. 1 shows the outcomes of the 35 courses of treatment completed at the time of submission, of which 18 of the 20 cases treated with the most effective dose and formulation achieved durable, measurable and clinically relevant tolerance.

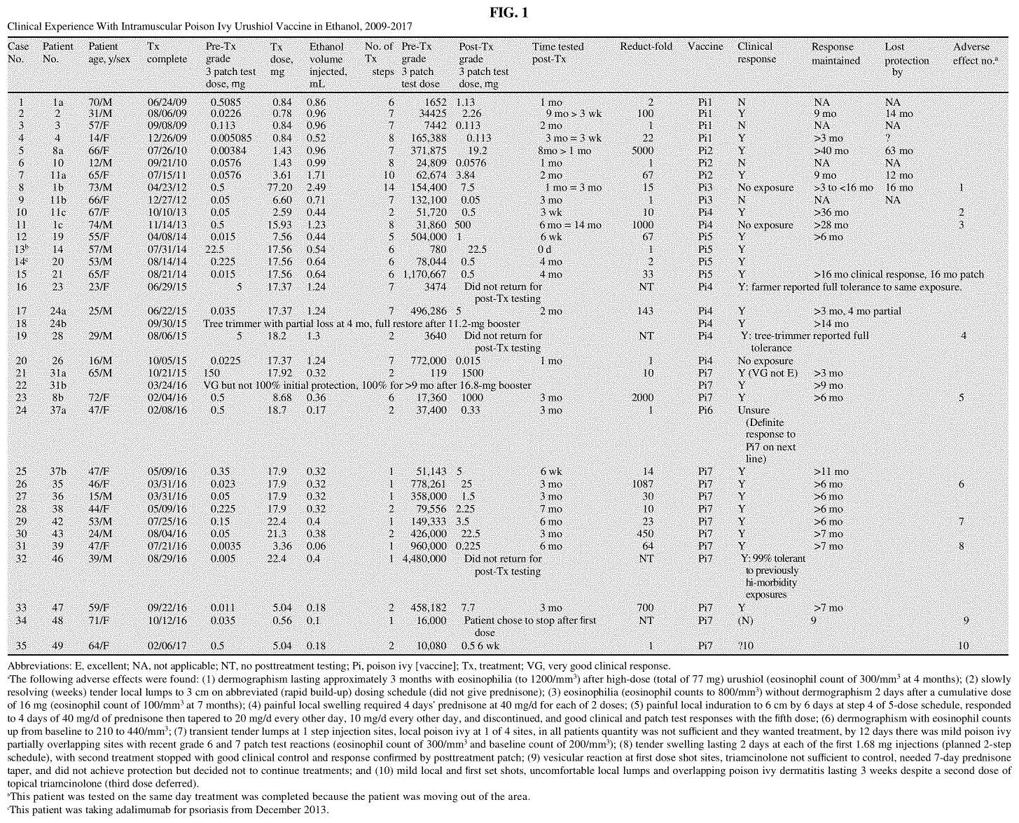

FIG. 2, Molecular sizing gel showing the difference in molecular size distribution produced by reaction of Ara h2 with different aldehyde coupling reagents.

FIG. 3 is a gel showing increasing conversion of Ara h2 to allergoid with increasing concentrations of a proprietary coupling reagent.

FIG. 4 shows reduced binding of the above allergoid to rabbit anti-Ara h2 by Western Blot.

FIG. 5 shows the same reduced binding measured by ELISA.

### DETAILED DESCRIPTION

Provided herein are methods of populating a target tissue of interest of a subject with particles of a modified or synthesized substance of interest, by precipitation, when the unmodified/unsynthesized form of the substance does not innately have the requisite solubility properties that would result in particle precipitation. The present methods include synthesis and/or modification of a substance using the application of any combination of one or more methods of solubility modification available to those skilled in the art, to enable substances that could normally not be distributed within the body of a designated target tissue by precipitation to be delivered to such tissues where they may confer therapeutic outcomes as unique to that method of tissue delivery as those demonstrated for a therapeutic substance that happens to have those solubility properties innately. Candidate substances of interest are any that can be modified to achieve insolubility in water and tissue fluid while retaining or achieving the solubility of target doses in small enough volumes of a pharmaceutically acceptable water-miscible solvent to precipitate into functionally sized insoluble particles upon administration. Successful candidate substances will be those for which developers can choose methods of physical and/or chemical modification that do not impair the physiologic or pharmaceutical ability of such modified substances to perform their intended functions following tissue delivery by precipitation.

The aspects, advantages and/or other features of example embodiments of the present disclosure will become apparent in view of the present detailed description. It should be apparent to those skilled in the art that the described embodiments of the present disclosure provided herein are merely exemplary and illustrative and not limiting. Numerous embodiments of modifications thereof are contemplated as falling within the scope of the present disclosure and equivalents thereto. Unless otherwise noted, technical terms are used according to conventional usage. All patents and publications mentioned in this specification are indicative of the level of those skilled in the art to which the invention pertains. All publications, patent applications, patents, and other references mentioned herein are incorporated by reference in their entirety.

In describing example embodiments, specific terminology is employed for the sake of clarity. However, the embodiments are not intended to be limited to this specific terminology.

As used herein, “a” or “an” may mean one or more. As used herein, “another” may mean at least a second or more. Furthermore, unless otherwise required by context, singular terms include pluralities and plural terms include the singular.

The term “antigen” as used herein is a substance capable of eliciting a specific response by the immune system of an exposed animal or human. An antigen is an allergen when the specific immune response is the development or modification of an allergy to that substance.

As used herein, the terms “solution”, “composition”, “therapeutic composition”, “immunotherapy composition” and “formulation” are used interchangeably herein and refer to a combination of elements that is presented together for a given purpose and in the physical form of a homogeneous solution. Such terms are well known to those of ordinary skill in the art.

The terms “substance of interest”, “active ingredient”, “therapeutic agent”, and “drug” are used herein to include any substance, drug or active ingredient that may be included in the present compositions for treating subjects, including, but not limited to, mammals. In particular, the present invention is directed to substances of interest that are modified or synthesized to impart particular solubility qualities to achieve the desired effect of precipitating in a target tissue. The solubility properties of the modified or synthesized substance, are of being insoluble in water but sufficiently soluble in a pharmaceutically acceptable, water-miscible non-aqueous solvent such that doses of the modified or synthesized substance of interest can be administered to target tissues of a subject in small enough volumes of the solvent, to be diluted rapidly enough by the water content of the target tissue to precipitate the substance in particles spanning an effective size range to achieve a desired physiological or pharmacological effect.

The present disclosure recognizes the numbers and types of substances of which particles can be deposited within the bodies of target tissues-of-interest by precipitation as water-miscible solvents in which they are administered are diluted by the water content of those target tissues following administration. This is accomplished by using known methods of solubility modification to confer the properties of insolubility in water while retaining or conferring solubility in a pharmaceutically acceptable water-miscible solvent on candidate substances that do not innately have those solubility properties.

As indicated above, previously poison ivy allergy vaccines included ether extracts of fresh or dried leaves evaporated to dryness and redissolved in sterile vegetable oils for either oral dosing or subcutaneous injection. In developing the present methods, the inventors chose to extract urushiol in ethanol instead of ether and to keep the active ingredient in ethanol instead of transferring it to sterile vegetable oils because ethanol is self-sterilizing and could be worked with on an ordinary clean laboratory bench. The inventors injected it into muscle instead of under the skin because ethanol is a tissue irritant and small volumes injected into muscle would be very rapidly diluted by the water content of muscle.

The inventors were pleasantly surprised to discover that poison ivy vaccines prepared and delivered in this way were unexpectedly more effective at inducing tolerance than anything previously reported. By enriching crude ethanol extracts of fresh leaves with additional concentrated urushiol they were able to achieve durable, measurable tolerance in 90% of treated patients.

To achieve immunomodulation whether from sensitization to tolerance or from tolerance to sensitization one must get the antigen to which one wants to modulate the response into antigen-processing cells of appropriate lineages. Those cells must then present the antigenic epitopes of those antigens to appropriate populations of T-lymphocytes in a cytokine milieu that favors immunomodulation in the desired direction.

When a water-insoluble substance dissolved in a small volume a water-miscible solvent is delivered into a non-liquid tissue in which the solvent is diluted by the available water content of that tissue, the substance becomes insoluble and precipitates as it loses solubility. The more rapidly this process of dilution takes place, the larger the number and smaller the size of the resulting particles. Size matters in that particles of different sizes can find their way into different types of cells by different mechanisms. Particles in the size range of 0.5 to 5 microns in diameter are very efficiently taken up by naïve antigen-processing cells by a process called macropinocytosis.

It may be helpful to understand the function of the immune system in different body tissues by thinking of it as a police force deploying under policies selected by thousands of generations of evolution. In internal tissues such as muscle there are normally very few threats, so the major function of the police force is to reinforce the maintenance of tolerance of one's own body tissues. Antigen-processing cells in muscle are primarily of tolerogenic lineages, there are ample numbers of tolerogenic T-regulatory lymphocytes to which they present antigenic epitopes to induce the development of immunological tolerance, and there is a tolerogenic cytokine milieu to support the process of tolerogenic immunomodulation.

It appears that the method of substance or vaccine delivery the present inventors developed, serendipitously hit a sweet spot of precipitated particle size in a microenvironment predisposed to immunomodulation from sensitization to tolerance. If the poison ivy urushiol content of one injection on a dosing schedule the inventors found to be effective precipitated totally as particles with a diameter of 2 microns, each such injection would deliver 0.6 billion such particles.

The skin, on the other hand, is an outside surface through which the body may be exposed to threats, and at which the police force has to guard against intruders. The skin is known to be a portal to the body that favors allergic sensitization. If one wants to achieve immunomodulation from a state of sensitization to a state of tolerance, one might want to precipitate close to a billion particles in the 0.5 to 5 micron size range in skeletal muscle. On the other hand, if one wanted to immunomodulate from tolerance to sensitization against autologous tumor antigens in cancer or from absence to presence of protective sensitization against such infectious diseases as malaria, HIV, Ebola, Zika, epidemic influenza and others, one might want to precipitate particles of antigen in the same size range in the dermis. One might also want to choose a solvent capable of penetrating phospholipid membranes both as a way to deliver the antigen into the dermis without injection and because such a solvent could access intracellular as well as extracellular water for dilution.

However, most substances-of-potential interest for precipitation by solvent dilution do not naturally have the requisite solubility properties of being insoluble in water but sufficiently soluble in a pharmaceutically acceptable water-miscible solvent that therapeutic doses can be dissolved in sufficiently small volumes of such a solvent that when those doses are administered to a non-liquid target tissue of interest the substance-of-interest will precipitate into particles of a size distribution appropriate to accomplish a therapeutic purpose that could not be accomplished by other available methods of tissue delivery.

The present invention includes methods to confer those solubility properties on many substances that do not innately have them and enable them to be deposited in designated non-liquid target tissues by precipitation as a water-miscible solvent in which they are administered is diluted by the water content of the target tissue, as well as methods of using such modified or synthesized substances to populate a target tissue with precipitated particles of the substance of interest, which may be for example, therapeutic agents.

Solubility can be modified by the use of coupling reagents that alter the boundary layer energy balance between the surface of the substance-of-interest and hydrophilic and hydrophobic microenvironments without changing its underlying chemical structure. The purpose of this principle is to minimize the risk of a coupling reaction made for the purpose of achieving the solubility properties needed for tissue delivery by precipitation from adversely affecting the ability of the modified substance from performing its intended function after tissue delivery by precipitation. Some coupling reactions will inevitably impact certain functions of many coupled substances. By providing a variety of coupling reagents that achieve the same solubility change but do so by binding to different reactive sites on a substance to be modified, however, provided herein are ways to meet the solubility goals of the present invention that avoid disrupting critical functions for many even if not all potential substances of interest.

Alternatively and/or supplementally, solubility can be modified by synthesizing modified versions of substances of interest that either already incorporate solubility-modifying hydrophobic surfaces or that have additional or preferential binding sites for solubility-modifying coupling reagents.

The inventors' discovery of the consequences of their inventive method of delivery of a substance of interest to a tissue of interest led to the identification of general principles by which substances having specific physical and solubility properties may be similarly deposited by precipitation into small particles in tissues of interest in which their presence as large numbers of small particles populating the bodies of those tissues may facilitate otherwise unachievable outcomes.

Tissues and intact organisms may respond differently to substances deposited in non-liquid tissues in the form of insoluble particles, than if the same substances are administered to the same tissues as solutions. They may also respond differently if millions of micron-sized particles are distributed within the substance of those tissues than if the same substances are deposited by injection of non-water-miscible suspensions that remain in macroscopic clumps. Accordingly, the inventors' discovery of methods of depositing insoluble particles of substances of interest in tissue, is groundbreaking and may be used in a wide variety of ways.

The present application utilizes the discovery made with regard to the deposition of urushiol by administering it in a solution of ethanol, as a method to deposit insoluble particles of any of a wide variety of substances of interest, which do not as they occur in nature innately have, but can be modified or synthesized to give them, appropriate solubility properties without significantly degrading their ability to perform their intended functions after precipitation in their intended target tissues-of-interest. The target tissue of interest will generally be a non-liquid tissue such as muscle or dermis. For example, the present methods include precipitating particles of a water-insoluble substance-of-interest in a non-liquid tissue that has sufficient structure to retain the precipitated particles in the locations at which they are formed, allowing them to interact with the recipient in the particle sizes and from the locations in which they are formed as the solvent is diluted by/the water content of the tissue.

In particular, example embodiments of the present invention include methods of populating a target tissue of interest of a subject with precipitated particles of a modified or synthesized substance of interest. Such methods may include precipitating within the target tissue said particles of the modified or synthesized substance of interest, wherein said modified or synthesized substance of interest does not, as it exists in nature in its unmodified and unsynthesized form, have properties of being insoluble in water but sufficiently soluble in a pharmaceutically acceptable, water-miscible non-aqueous solvent, such that doses can be administered to target tissues in small enough volumes of the pharmaceutically acceptable, water-miscible non-aqueous solvent to be diluted rapidly enough by the water content of the target tissue of interest to precipitate in particles spanning an effective size range to achieve a desired physiological or pharmacological effect. The modified or synthesized substance of interest is either a modified substance, which is modified from a substance's original form to impart solubility properties not present in an original, unmodified form of the substance, or it may be a synthesized substance that is synthesized in such a way to confer solubility properties. In example methods, the precipitating takes place upon administering a solution of the modified or synthesized substance of interest to the subject; in which the solution includes a pharmaceutically acceptable, water-miscible non-aqueous solvent, and the modified or synthesized substance of interest that is soluble in the pharmaceutically acceptable, water-miscible non-aqueous solvent, and that is insoluble in tissue fluids of the target tissue of interest. The pharmaceutically acceptable, water-miscible non-aqueous solvent is freely miscible with the tissue fluids of the target tissue of interest. In example embodiments, the solution is administered to the tissue of the subject in a volume of the pharmaceutically acceptable, water-miscible non-aqueous solvent such that the solution will be diluted by tissue fluids in the target tissue of interest following administration, at a rate that results in precipitation of an effective mass of the modified or synthesized substance of interest with a distribution of particle sizes that will accomplish said desired physiological or pharmacological effect for which the modified or synthesized substance of interest is administered.

Further example embodiments include methods of populating a target tissue of interest of a subject, with precipitated particles of a modified or synthesized substance of interest, which include administering a solution to a target tissue of interest of a subject in which the solution includes a pharmaceutically acceptable, water-miscible non-aqueous solvent, and the modified or synthesized substance of interest. In such methods, the modified or synthesized substance of interest is soluble in the pharmaceutically acceptable, water-miscible non-aqueous solvent and insoluble in tissue fluids of the target tissue of interest; and wherein the pharmaceutically acceptable, water-miscible non-aqueous solvent is freely miscible with the tissue fluids of the target tissue of interest. In example embodiments, the solution is administered to the target tissue of interest of the subject in an effective volume of the pharmaceutically acceptable, water-miscible non-aqueous solvent, such that the pharmaceutically acceptable, water-miscible non-aqueous solvent will be diluted by tissue fluids in the target tissue of interest following administration to the tissue, at a rate that results in precipitation of an effective dose of particles of the modified or synthesized substance of interest in the target tissue of interest with a distribution of particle sizes of the substance of interest that will accomplish a purpose for which the modified or synthesized substance of interest is administered. As described herein, the modified or synthesized substance of interest is either a modified substance, which is modified from a substance's original form to impart solubility properties not present in an original unmodified form of the substance, or a synthesized substance that is synthesized in such a way to confer solubility properties; and the modified or synthesized substance of interest does not, as it exists in nature in its unmodified and unsynthesized form, have properties of being insoluble in water but sufficiently soluble in a pharmaceutically acceptable, water-miscible non-aqueous solvent such that doses can be administered to target tissues in small enough volumes of said pharmaceutically acceptable, water-miscible non-aqueous solvent to be diluted rapidly enough by the water content of the target tissue of interest to precipitate in particles spanning an effective size range to achieve a desired physiological or pharmacological effect.

In example embodiments, the tissues may be for example, tissues in which it would be safe to precipitate particles of water-insoluble therapeutic agents in situ with precipitated particles of a modified or synthesized substance of interest, in which tissue the precipitated particles remain where they are formed for a sufficient time to begin their interaction with the physiology of the recipient in the physical state and locations in which they were formed.

Example target tissues of interest should be understood to be a non-liquid tissue such as muscle or skin or the tissues underlying the body's various mucus membranes. For example, the present methods include populating a non-liquid target tissue with sufficient structure that molecules of a water-insoluble solute administered to that tissue in small volumes of a water miscible solvent will be sufficiently retained by that structure to precipitate in situ or in place within an area of the body of the tissue as the solvent is diluted by the water content of the tissue.

In example embodiments, the precipitated particles remain in the target tissue where formed.

The solubility requirement of this method is that the modified or synthesized substance of interest must be soluble in a pharmaceutically acceptable water-miscible solvent (generally either or both of ethanol and dimethylsulfoxide (DMSO), but not limited thereto) and insoluble in tissue fluids and/or water (noting that insolubility in water which is known for many candidate substances-of-interest may be used e.g. as a surrogate to predict insolubility in tissue fluid: Certain proteins that may be candidates for tissue delivery by precipitation may be SOLUBLE in water but insoluble at the salt concentrations present in tissue fluid. There is NOTHING to the inventors' knowledge, however, that is insoluble in water but will be soluble in tissue fluid.

According to non-limiting example embodiments, any substance of interest may be administered that does not naturally have these solubility properties, but may be modified or variant forms specifically synthesized to have these solubility properties. A further requirement for successful implementation of this invention will be that effective doses of the substance-of-interest can be dissolved in a sufficiently small volume of such a solvent to be diluted by tissue fluid in the target tissue of interest at such a rate that the substance of interest will precipitate in particles of appropriate number and size to achieve the purpose of administration.

In the fields of allergy and immunology, vaccines act by being taken up by or otherwise finding their way inside of antigen-presenting cells (APC's) that then present their allergenic structures called epitopes to appropriate populations of T- and possibly also B-lymphocytes. The present invention provides an extremely efficient way for vaccines that do not otherwise have the necessary solubility properties for delivery by precipitation to be delivered to the APC's that populate physiologically appropriate target tissues. As the history of technology is that when a new way of doing something is found to be useful in one field, others find useful ways to apply it elsewhere. The inventors thus believe that the present invention of a novel way to precipitate particles of substances of interest in non-liquid tissues of interest by giving the requisite solubility properties to substances that do not innately have them, will find application in other fields, as well.

Accordingly, in view of the above, the present application is directed to expanding the range of substances capable of being delivered to designated target tissues by precipitation from pharmaceutically acceptable water-miscible solvents to substances that do not innately have the requisite solubility properties as they exist in nature. These properties can be conferred by either chemical modification or by synthesis of non-natural molecular forms specifically designed to have the properties of being insoluble in water but sufficiently soluble in a pharmaceutically acceptable water-miscible solvent to be able to deliver effective the doses by precipitation in effective ranges of particle size upon administration to designated target tissues. The modified or synthesized substance of interest must be soluble in a pharmaceutically acceptable, water-miscible solvent but insoluble in tissue fluids of the target tissue of interest; and the pharmaceutically acceptable solvent must be freely miscible with the tissue fluids of the target tissue of interest.

The modified or synthesized substance-of-interest is administered in a volume of the pharmaceutically acceptable solvent that will be rapidly diluted by tissue fluids in the target tissue of interest following administration. The size distribution of precipitated particles will depend on the rate at which the solvent is diluted, and in some applications may be varied by changing the volume of solution administered and the injection speed if injected.

Changing the site of administration may change the volume of tissue fluid functionally available to dilute it. Increases in the viscosity of injected solutions will slow dilution and result in the precipitation of smaller numbers of larger particles. The presence of other molecular species may either increase speed of precipitation yielding larger numbers of smaller particles or slow it yielding smaller numbers of larger particles.

There will be combinations of substance, solvent and target tissue for which desired particle size distributions may not be achievable but with the information contained herein those skilled in the art should be able to optimize the listed parameters to the greatest extent possible for each individual application.

According to the present methods and solutions, the substance of interest may include for example an allergen administered for the purpose of tolerance induction, a vaccine for the purpose of inducing sensitization, a drug, pro-drug or other therapeutic agent for which tissue deposition of particles will result in a desired time course of drug release and drug action or a drug, pro-drug or other therapeutic agent for which tissue deposition of particles will result in uptake by specific cell populations. The suitable solvent may include for example, a pharmaceutically acceptable non-aqueous solvent, including, but not limited to one of more of ethanol, ethyl acetate, acetonitrile and dimethylsulfoxide (DMSO). Other solvents either presently known or developed de novo in the future may be designated pharmaceutically acceptable for specific circumstances of use (as the inventors have shown to be the case for ethanol injected in small volumes into muscle).

According to the present invention, the solution of a modified or synthesized substance of interest may be administered to a subject (such as a mammal or other animal) by injection, for example into muscle or into other non-liquid tissues. The substance of interest may be administered to the target tissue transdermally if the solvent is capable of carrying it across the epidermal barrier as will be the case for many potential substances-of-interest dissolved in dimethylsulfoxide. The above examples are not limiting as it would be apparent to those skilled in the art based on the present disclosure, other methods may effectively deliver specific substances for precipitation in specific target tissues under specific circumstances. If the solvent is capable of diffusing across phospholipid membranes without provoking a prohibitive irritant response the solution may be administered topically to mucus membranes of the respiratory tract including the conjunctiva, the gastrointestinal tract, the urinary tract and either locally or generally onto the peritoneum.

According to non-limiting example embodiments, substances of interest may be modified or synthesized natural or recombinant autologous antigens and their derivatives, which may be administered for example for the treatment of autoimmune diseases, as well as other substances. For example, the substance of interest may include wherein the modified or synthesized substance of interest comprises modified allergens, modified and natural synthetic allergens, modified tumor antigens, and synthetic tumor antigens.

According to non-limiting example embodiments, the present methods may be essentially “atraumatic” in comparison to any other known way to populate a tissue with insoluble particles of a substance of interest. There may be a momentary irritant effect indicated by the pain of ethanol injection but for the small volumes the inventors inject into muscle in which it is rapidly diluted there does not appear to be any long term damage such as that deliberately produced by the published medical use of ethanol by injection in volumes and into tissues in which it is not rapidly diluted and in which its prolonged irritant effect results in tissue destruction and scarring.

The manipulations needed to administer the volumes of solvent needed to deliver the water-insoluble substance of interest to the target tissue of interest will inevitably cause some physical and/or chemical trauma to that tissue, but if properly chosen that trauma will be minimal compared to any other known method of depositing large numbers of small particles within the substance of those tissues. This method of substance deposition within a tissue is therefore “relatively atraumatic.”

The choice of solvent and of initial preparation of the substance of interest may depend on the tissue and species of recipient. The need to control and minimize the water content in the course of vaccine processing is important for compositions of the modified or synthesized substances-of-interest of the present invention.

The present application further relates to methods of making solutions and compositions disclosed herein.

Potential uses for the present invention include:
1. Administration of allergy vaccines to induce immunologic tolerance in both humoral and cell-mediated allergy.
2. Administration of vaccines against infectious diseases to induce a protective immune response.
3. Administration of tumor vaccines to sensitize patients against their cancers.
4. Administration of tissue antigens or surrogates for tissue antigens to induce tolerance in autoimmune disease.
5. Administration of any other therapeutic or pharmaceutical substance for any purpose for which deposition of insoluble particles of the substance within the tissue by precipitation might facilitate the achievement of a desired outcome. Non-limiting examples include deposition of particles of a size that will give a desired pattern of time-release by dissolution, deposition of particles of a drug in a tissue and size range that favor uptake by specific cell populations in the manner that allergy vaccine particles in the size range of 0.5 to 5 microns in diameter are favorably taken up by antigen-processing cells, and deposition of particles of pro-drugs designed to be activated and released by the occurrence of specific physical or chemical events within those tissues.

Factors which can affect the number and size distribution of the resulting particles may include:
1. Viscosity of the administered solution.
2. Volume of solution administered.
3. Volume of tissue fluid immediately available to dilute administered solvent.
4. Rate of replenishment of reservoir of tissue fluid locally available to dilute administered solvent.

Particle sizes in the 0.5 to 5 micron diameter range are likely to be optimal for the other modifications of immune response in the above “Potential uses” list, as well as for immunomodulation from sensitization to tolerance. Experience to date suggests that particle size distributions that are efficiently taken up by antigen-processing cells for modification of the immune response can be achieved if the viscosity of solutions administered by injection is either less than or not significantly greater than the viscosity of water or of the tissue fluid with which it will be mixed upon delivery.

According to non-limiting example embodiments, dimethylsulfoxide (DMSO) may be an effective solvent for topical administration to skin and/or mucus membranes. The ability of DMSO to diffuse across cell and tissue membranes that are impermeable to ethanol it may let it access intracellular as well as extracellular water for dilution and enable effective precipitation of particles in tissues that do not have the extracellular water content needed to dilute injected solutions in ethanol rapidly enough to achieve precipitation in a target range of particle size.

As a starting point for determining suitable solutions in view of the present disclosure, one may use a solution of the lowest possible viscosity for desired dose of the substance of interest, administered in the smallest possible volume, and into the largest possible volume of readily available tissue fluid. The relatively high viscosity of DMSO is not expected to be a complication for transdermal or trans-mucosal administration, as while its viscosity will slow its rate of penetration it will not slow the rate of dilution by available tissue water of the DMSO that has already penetrated into the tissue.

Also provided herein are immunotherapy compositions and methods that provide immunotherapy for inducing a state of immunologic tolerance to an allergen. Such tolerance may act to reduce and/or prevent future allergic reactions in a subject (such as mammals), which allergic reactions are caused by exposure of the subject to an allergen. By way of example, present embodiments include compositions and methods of treating a subject by administering compositions to the subject, wherein the compositions include one or more modified, synthesized or recombinant allergens and/or their derivatives and/or precursors including but not limited to peptide and nucleic acid allergy vaccines and at least one pharmaceutically acceptable non-aqueous solvent. The at least one active ingredient is soluble in the solvent and insoluble in water and interstitial fluid. The solvent is miscible with water and interstitial fluid. Also included are methods of making the present immunotherapy compositions, and methods of administering these compositions to a subject. Thus, the present compositions and methods may be useful for example, in reducing or eliminating a patient's allergic response to exposure to an allergen such as those in peanuts, stinging insect venoms, latex, tissue, and the like.

Various formulations and methods for allergen immunotherapy have enabled the generally safe and effective induction or enhancement of immunologic tolerance in numerous allergic conditions. These treatments are sub-optimal or not available for many allergic diseases, however. The present inventors have discovered that when an allergen is formulated with a suitable solvent as set forth herein and administered as set forth herein, the allergen has unexpectedly superior effects inducing a state of immunologic tolerance to the allergen. Thus, the present compositions and methods provide a previously undiscovered and safe method of treating an allergic patient.

According to other non-limiting embodiments, the method of the present invention may be used to induce immunomodulation either from sensitization to tolerance or from tolerance to sensitization. Non-limiting example embodiments of modified natural, recombinant or synthesized allergens and their derivatives that may be included in the formulation include for example, plant allergens that do not naturally have the necessary solubility properties, food allergens including but not limited to those of peanut, milk, egg, tree nuts, fish, shellfish, wheat, and soy); insect-derived allergens; inhalant airborne allergens (e.g., to treat respiratory allergies); contact allergens; latex allergens; chemical and biological allergens (which cause e.g., allergic contact dermatitis), tissue allergens, drug allergens; modified versions of such allergens, and synthetic equivalents of such allergens. According to other example embodiments, “active ingredients” may also include one or more additional or different ingredients than those listed above, such as one or more other allergens, other modified allergens, non-allergens or synergistic ingredients for which deposition within a designated target issue of a mammal or other subject (such as avian) may achieve a desired effect.

Encompassed by the terms “substance of interest”, “active ingredients” and/or included within the meaning of “allergen” as provided herein, are modified and/or synthesized substances of interest, such as modified allergens and both modified natural and synthetic allergens, or any other variation of any allergens, or similar ingredient that may provide the same or similar active components as the indicated active ingredient. According to non-limiting example embodiments, active ingredients may include one or more normal or abnormal tissue antigens, such as modified and/or synthesized tumor antigens or their derivatives for immunomodulation from tolerance to sensitization in patients with cancer, and modified and/or synthesized normal tissue autoantigens or their derivatives for immunomodulation from sensitization to tolerance in patients with autoimmune diseases. “Active ingredients” may also be drugs or other substances whose intended effect is other than the modifications of immunologic reactivity for which this method was initially developed. The modified or synthesized substances of interest herein, are intended to include modified or synthesized substances of interest that are soluble in a pharmaceutically acceptable, water-miscible, non-aqueous solvent and insoluble in tissue fluids of a target tissue of interest; and for which those solubility properties are imparted by modification or synthesis of the substance. That is, in the present invention, the modified or synthesized substance of interest does not, as it exists in nature in its unmodified and un-synthesized form, have properties of being insoluble in water but sufficiently soluble in a pharmaceutically acceptable, water-miscible solvent, such that doses can be administered to target tissues in small enough volumes of said pharmaceutically acceptable, water-miscible solvent to be diluted rapidly enough by the water content of the target tissue of interest to precipitate in particles spanning an effective size range to achieve a desired physiological or pharmacological effect

“Solubility” as used herein, and as commonly known in the art, is the property of a solid, liquid, or gaseous chemical substance called solute to dissolve in a solid, liquid, or gaseous solvent to form a homogeneous solution of the solute in the solvent. The solubility of a substance fundamentally depends on the physical and chemical properties of the solute and solvent as well as on temperature, pressure and the pH of the solution. The extent of the solubility of a substance in a specific solvent is measured as the saturation concentration, where adding more solute does not increase the concentration of the solution and begin to precipitate the excess amount of solute.

The solubility of one substance in another is determined by the balance of intermolecular forces between the solvent and solute, and the entropy change that accompanies the process of solvation. Factors such as temperature and pressure will alter this balance, thus changing the solubility. Solubility may also strongly depend on the presence of other species dissolved in the solvent.”

According to non-limiting example embodiments throughout this application, the at least one substance of interest or pharmaceutically acceptable active ingredient, may be at least one of tissue or modified tissue antigens.

Recombinant DNA technology allows natural, modified or totally synthetic segments of genetic code to be inserted into organisms to which they are not native, usually specific strains of bacteria, which can be induced to produce any protein(s) coded by the transplanted genetic code. When such proteins are allergens, mutant versions which may have altered allergenicity and potential value as materials for the production of allergy vaccines can be produced by such measures as exposing their carrier bacteria to chemical mutagens or ionizing radiation. Allergens produced by recombinant DNA technology will be termed “recombinant allergens” whether or not their DNA has been modified from that of the natural allergens, and recombinant allergens which are further modified by physical or chemical methods after synthesis will be termed “modified recombinant allergens.” Any or all of these as well as allergens produced by methods to be developed in the future may be candidates for enhancement of their ability to achieve therapeutic immunomodulation by the present invention.

The term “excipient” is used herein to include pharmaceutically acceptable inert substances added to a drug formulation or composition to give e.g., a desired consistency or form, or used as a carrier. Non-limiting examples of excipients that may be included in the present compositions and/or formulations herein may include, but are not limited to binders, fillers, diluents, lubricants, anti-infectious agents, antimicrobial agents and solubility modifiers, and other excipients known to those skilled in the art, depending e.g., on the composition being formed, intended method of administration and/or method of formation, the active ingredient(s) being used, etc. When using excipients however, one must keep in mind that traditional excipients may alter the solubility of the substance of interest in the chosen solvent and its speed of dilution on exposure to tissue fluid, thereby altering the suitability and effectiveness of the present compositions and methods. For example, a viscous injected water-miscible solution may equilibrate with (and be diluted by) available tissue water more slowly than a less viscous solution. The slower rate of dilution will give molecules of solvent more time to fine each other and coalesce into smaller numbers of larger particles with a smaller total surface area that ties up less boundary layer energy. Accordingly, typical excipients, which in other formulations may be used to increase viscosity, may be unsuitable for the present compositions of this invention.

As used herein, “an effective amount” refers to an amount of the specified constituent in a solution or composition, or an amount of the overall solution or composition that is effective in attaining results, the purpose for which the constituent or composition is provided. Therefore, an effective amount of a composition would be an amount suitable for achieving the desired immunotherapy effects in a subject, such as a mammal (e.g., human) to which the composition is administered.

The terms “interstitial fluid” and “tissue fluid” as used herein are intended to have their common meaning in the art. In particular, “interstitial fluid” (or tissue fluid) is a solution that bathes and surrounds the cells of multicellular animals. Insolubility in water is cited as a surrogate marker of insolubility in tissue fluid because solubility in water is either already known or else easy to measure for most candidate substances of interest, and insolubility in water is essentially a guarantee of insolubility in tissue fluid.

As used herein, the term “recipient”, “subject” or “patient” is intended to include any animal, such as a mammal (including, but not limited to humans) to whom the present compositions may be administered. A subject or patient may or may not be under current medical care, and may or may not have had one or more prior treatments. Although, as would be apparent to those skilled in the art, the formulations and dosages may be different for non-humans than for humans, taking into consideration certain solvent requirements are provided herein for safety for injection.

Additional example embodiments, the present invention (solutions, compositions, methods, etc.) may include solutions, compositions and methods for the treatment of birds. Accordingly, example embodiments are directed to compositions and methods for avian use as well.

A target tissue for this invention also need not be a part of an intact organism at the time. Examples of this would include but not be limited to treatment of tissues in organs removed from living or cadaver human donors being prepared for transplantation, and treatment of human or animal organs to prepare them for various forms of research which in the case of animals either could or could not precede organ or tissue transplantation.

According to example embodiments, the solution as well as the isolated solvent should be miscible with water. It would be apparent to those skilled in the art that solutions relevant to veterinary use and determined by appropriate regulatory authorities to be safe and effective for such use may be different than vaccines relevant to and determined to be safe and effective for human use. Also, solvents and any other additives determined to be pharmaceutically acceptable for veterinary use may differ from those determined to be pharmaceutically acceptable for human use.

Example embodiments are directed to immunotherapy compositions that include at least one pharmaceutically acceptable active ingredient selected from the group consisting of modified natural, recombinant and synthetic allergens and all of their derivatives and at least one pharmaceutically acceptable non-aqueous solvent. The solvent is miscible with water and interstitial fluid. The at least one active ingredient is insoluble in water and in interstitial fluid (e.g., of the subject or patient to whom the composition will be administered), and soluble in the solvent.

The solutions, compositions and/or methods herein may also be used e.g., for the immunotherapy treatment of autoimmune disorders in subjects including but not limited to mammals.

According to non-limiting embodiments, the compositions herein may be used as a booster of a tolerogenic immune response in diseases of pathological immunological sensitization or of a sensitizing immune response in diseases of pathological immunological tolerance (as in many cancers) or of suboptimal protective sensitization (as in epidemic and individually hard-to-control infectious diseases).

According to non-limiting example embodiments, solutions or compositions provided herein may include one or more substances of interest, or active ingredients, and a non-aqueous solvent. According to non-limiting example embodiments, as discussed herein, the substance of interest or active ingredient may be at least one modified natural, recombinant or synthetic allergen or derivative, selected from plant allergens; food allergens (e.g., peanut); insect-derived allergens (e.g., hymenoptera venoms); inhalant airborne allergens; contact allergens; latex allergens; chemical and biological allergens including tissue allergens; drug allergens; modified versions of such allergens, including derivatives, and synthetic equivalents of such allergens. According to further non-limiting example embodiments, the allergen may comprise one or more of the following, plant allergens; non-plant food allergens; insect venom allergens; latex allergens and tissue allergens, which may be modified natural, recombinant or synthetic, and which include derivatives or modifications thereof.

Derivatives or modified allergens within the scope of the present invention may include for example “allergoids” made by polymerizing protein allergen molecules together and peptide vaccines, made by breaking them apart. Both have been more effective at inducing tolerance than their unmodified precursors. To date neither has been studied by precipitation in muscle and both are potential candidates for the present invention. Nucleic acid vaccines, strings of DNA or RNA containing the genetic code for protein allergens, are also candidates for feeding to antigen-processing cells by precipitation.

Latex is a contact allergen capable of causing the same type of allergic contact dermatitis produced by poison ivy. It is different and essentially in a class by itself in its ability to first elicit poison ivy-like allergic contact dermatitis and then (with continuing exposure to the increased numbers of allergen processing cells in the cytokine milieu of the allergic contact dermatitis reaction) stimulate an IgE anaphylactic humoral antibody response, like that of severe food or insect sting allergy.

The methods and solutions of the present invention may also be used to treat individuals allergic to stinging insect venoms or having other insect related allergies, for example, by administering the present compositions, including “insect-derived allergens” (such as insect venom allergens) to an individual having insect sting or insect bite allergy. Hymenoptera is the order of Class Insecta that includes sawflies, wasps, bees, and ants. The methods of the present invention may enable improvements in the treatment of allergies to biting as well as stinging insects.

Many commonly encountered substances including both proteins (such as latex and stinging insect venoms) and small molecules (such as poison ivy urushiol) are allergenic (capable of inducing allergic reactions) with sufficient exposure in genetically susceptible individuals. For many of these allergens VDBP (vaccine delivery by precipitation) has the potential to induce immunological tolerance for which there is presently no safe and effective alternative method. For others, VDBP offers the potential to improve some or all of safety, effectiveness, patient and health care provider convenience, and savings in total cost of health care. The majority of these allergens will not innately have the necessary solubility properties for VDBP. The present invention encompasses methods to give them those solubility properties and having done this to administer them by VDBP. For substances-of-interest that are not allergy vaccines a more appropriate term for the method might be “Substance delivery by precipitation” or SDBP.

Pharmaceutically acceptable non-aqueous solvents according to the present invention may include for example one or more solvents selected from the group consisting of ethanol, ethyl acetate, acetonitrile, dimethylsulfoxide (DMSO), and other water-miscible solvents. The term “non-aqueous solvent” as used herein is something that isn't water and that because it has different properties of molecular stabilization and surface energy than water is capable of dissolving various substances of interest that are insoluble in water. With respect to ethanol, for example, different water-insoluble substances may remain soluble up to different fractions of water in their microenvironment. Some may require 100% ethanol to remain soluble in effective concentrations for tissue delivery by the method of this invention. Some may remain soluble in 95% ethanol/5% water. Some may remain soluble when the ethanol/water ratio is 70%/30% or even less but still precipitate as particles with functional particle size distributions if those solutions are rapidly diluted to near-zero ethanol concentration by injection of small volumes into tissues with sufficient water content for rapid dilution. The relevant physical chemistry is that as the water content of the composition increases by either inclusion of more water in the composition or as the composition is diluted by tissue water following administration, that water will increasingly confer the surface energy properties of water onto the resulting mixture, reducing the solubility of its water-insoluble solutes and leading to precipitation at levels of micro-environmental water content that may vary for different substances-of-interest.

The amount and type of solvent may depend on both the substance to be dissolved and the intended route of administration. At the time of filing of this application, DMSO is the only solvent approved for human use that has the ability to carry dissolved substances across phospholipid membranes including those of the skin, the conjunctiva and the lining membranes of the upper and lower respiratory, digestive and urogenital tracts.

Additionally, according to non-limiting example embodiments the solvent may need to have a low viscosity to achieve rapid equilibration with and dilution by the water content of interstitial or tissue fluid. Viscous water-miscible solvents such as glycerol may equilibrate with interstitial or tissue fluid so slowly that a dissolved substance-of-interest would precipitate in much smaller numbers of much larger particles, which are likely to have a much different effect or lack of effect than if administered in a solvent with lower viscosity.

According to non-limiting example embodiments, the solvent may be ethanol. When the inventors began this study the most concentrated (purest) ethanol available in pharmaceutical grade was 95% but anhydrous (100%) pharmaceutical grade ethanol is now commercially available. According to other non-limiting example embodiments, the solvent may be ethyl acetate or any other pharmaceutically acceptable water-miscible, non-aqueous solvent.

In referring to “pharmaceutically acceptable” solvents, the inventors intend to encompass (1) solvents that meet appropriate regulatory criteria for safe use in humans or animal recipients of vaccine or drug to be delivered by the present methods; and (2) solvents which are readily miscible with water and interstitial fluid, Interstitial or tissue fluid is a solution of certain water-soluble proteins, carbohydrates, salts and small quantities of other substances in physiologic saline

When and whether a particular solvent can be considered “pharmaceutically acceptable” will generally be determined by pharmaceutical regulatory authorities, depending for example on the type of solvent, intended dose, intended use and target tissue. For example, some solvents may be considered pharmaceutically acceptable in small amounts administered to certain tissues and as vehicles to deliver certain drugs for certain clinical indications. The same solvents may not be considered pharmaceutically acceptable for administration in larger amounts, to other tissues or for other clinical indications.

A substance of interest, vaccine, drug or active ingredient to be delivered should be soluble in the water-miscible solvent but insoluble in water and in the solution of proteins and carbohydrates in physiologic saline that comprises interstitial or tissue fluid. Throughout this application, it is indicated that the substance of interest must be insoluble in water and/or tissue fluid. Insolubility in water is a surrogate marker for insolubility in tissue fluid. The only substances for which solubility in water is likely to be different from solubility in tissue fluid are proteins, for many of which the salt content of tissue fluid makes than less soluble than in water. No potential substance of interest is known for which the other substances present in tissue fluid would increase its solubility compared to that in water. Water is a readily available standard solvent in which to determine the insolubility needed for use of this method while tissue fluid from the various tissues of interest is often much less readily available. Any substance and particularly any protein that is insoluble in water will almost certainly also be insoluble in tissue fluid (though the reverse is not necessarily true).

As discussed above, one or more pharmaceutically acceptable excipients may be used in the present compositions in accordance with the present application, for example for administration purposes and/or to help the allergen achieve desired properties, as long as the excipients do not adversely affect the physical chemistry of the interaction between the substance-of-interest and the microenvironment into which it is administered. Other potential pharmaceutically acceptable carriers or components may be added to the formulation if they facilitate the achievement of the solubility properties needed for effective target tissue delivery by precipitation.

Accordingly, example compositions may include one or more excipients that may be selected, for example based on the type of composition being formed, desired route of administration and properties to be achieved, etc. A goal of the method of this invention for most anticipated applications is to have the administered active ingredient become insoluble as quickly as possible, as the water-miscible solvent in which it is administered is diluted by interstitial &/or tissue fluids. The physical chemistry of solubility is such that a faster rate of solvent dilution yields a more rapid loss of solubility and precipitation into a larger number of much smaller particles than if solvent dilution takes place more slowly. Rapid solvent dilution and precipitation appear to be important for applications of this invention to immunomodulation. Accordingly, excipients that stabilize a substance of interest in the liquid phase or delay solvent dilution and precipitation are likely to be counterproductive for applications to immunomodulation. The same excipients could be useful, however, for applications that require precipitation of a substance of interest into smaller numbers of larger particles.

According to non-limiting example embodiments, the formulation or composition may be a combination formulation with more than one modified or synthesized substance of interest, of either the same or different type and class, and for treatment of either the same clinical condition or of different clinical conditions. Multiple allergens may be administered in a single dose if their preparation process and solvent requirements are mutually compatible.

The present compositions may further include one or more pharmaceutically acceptable active ingredients that are not the substance of interest, in addition to the substance of interest, solvent and/or additional ingredients. Such active ingredients may include for example other treatments for allergies or autoimmune disorders that act by mechanisms other than immunomodulation but are also effectively delivered by precipitation.

As previously indicated, the solubility properties of the present composition are of utmost importance in preparing a suitable formulation. Thus, compositions of the present invention may include the addition of adsorbents and/or coupling of side chains to a substance of interest, such as allergens or allergoids, to confer appropriate solubility properties. According to example embodiments, the at least one modified or synthesized water-insoluble active ingredient must be soluble in the pharmaceutically acceptable water-miscible in which it is intended to be administered and it must be able to precipitate in a non-liquid target tissue upon administration Additionally, the present application is intended to include other physical or chemical modifications known to those skilled in the art or that may be discovered in the future to alter the solubility properties of candidate substances-if-interest &/or their derivatives in view of the present disclosure. Tyrosine adsorption is an example of a non-covalent modification capable of changing the solubility properties of certain proteins including certain protein allergens.

According to non-limiting example embodiments the present compositions or formulations may be in e.g., a liquid formulation/solution suitable for injection, such as intramuscular, subcutaneous, intradermal, or intraperitoneal injection, or it may be in a formulation that is suitable for administration by other routes, e.g., topical or transdermal.

Although intramuscular (IM) formulations may be preferred for administration of certain substances of interest, particularly for human applications, it is possible that other routes of administration may be suitable as well. Intraperitoneal injection might be an effective method of allergen delivery in small laboratory animals. Other possible formulations may include liquids, powders, or other formulations that may be suitable for various types of injection or topical administration.

By way of example, a powdered formulation could be lyophilized and redissolved in a pharmaceutically acceptable solvent for injection. It is possible that some solvents, possibly DMSO, could diffuse away rapidly enough to precipitate functionally sized particles for certain applications following injection into the subcutaneous space. DMSO penetrates skin following topical application and has the potential to carry therapeutic doses of a dissolved substance-of-interest through the epidermis and into the dermis before the DMSO is diluted sufficiently for the substance it carries to precipitate.

It is possible that the present compositions and methods might be effective when administered in ways other than IM administration, for example, either by topical application in DMSO (for example) or by intradermal injection. According to example embodiments, such methods might be capable of inducing immunomodulation from tolerance to sensitization in cancer patients who are immunologically unreactive to their cancers. The mechanism would again be deposition of tens or hundreds of millions of 0.5 to 5 micron sized particles as the solvent is diluted by what in this case would be interstitial fluids of the dermis (deeper living layer of the skin) and possibly superficial subcutaneous tissue. This could work if the mechanism of action of urushiol tolerogenesis is not subcutaneous persistence of small insoluble particles but their uptake by antigen-processing cells of the immune system. In skin, where antigens are processed by a unique-to-skin set of antigen-processing cells called Langerhans cells, which are known in many circumstances to promote sensitization rather than tolerance, precipitation of antigen in the form of critically sized particles from solvent could be critical to the success of the method.

Vaccine delivery by precipitation in the dermis should be an efficient way to feed antigen to the same populations of antigen-processing cells that induce sensitization to poison oak and poison ivy in the genetically predisposed 85% of Americans who become sensitized following skin exposure to those allergens. Conditions in which it could be therapeutic to induce immunomodulation from a state of active immunological tolerance to a state of sensitization include the full range of various types of cancer, in which the methods of the present invention could improve the efficiency with which tumor antigens can be delivered to populations of antigen-presenting cells capable of effecting that therapeutic immunomodulation in the presence of the already-established state of immunological tolerance that enables cancer cells to survive. Conditions in which it could be therapeutic to induce immunomodulation from a state of lack of effective immune recognition to a state of active sensitization include both treatment and protective immunization against such infectious diseases as malaria and infection with HIV, Ebola, Zika, and epidemic strains of influenza.

The same increased efficiency of vaccine delivery to relevant populations of antigen-processing cells that achieved the world's first predictably, measurably and demonstrably effective immunomodulation from sensitization to tolerance for poison ivy might be equally effective at inducing immunomodulation from tolerance or unresponsiveness to sensitization in the fields of cancer and infectious disease. Most if not all candidate vaccines for these conditions do not innately have the necessary solubility properties for VDBP and are thus candidates for the methods of this invention.

As discussed above, the pharmaceutically acceptable active ingredient or substance of interest may include at least one ingredient selected from the group consisting of plant allergens; food allergens; insect-derived allergens; inhalant airborne allergens; contact allergens; latex allergens; chemical and biological allergens such as tissue allergens; drug allergens; modified versions of such allergens; and synthetic equivalents of such allergens.

According to further non-limiting example embodiments, methods of making a composition such as a solution herein (such as an immunotherapy composition) may include combining a modified or synthesized substance of interest, such as at least one pharmaceutically acceptable active ingredient selected from the group consisting of modified natural, recombinant, and synthetic allergens; with a pharmaceutically acceptable non-aqueous solvent to form a composition comprising the active ingredient and solvent. The composition is then formulated for administration (e.g., by injection, topical administration, etc) to a subject in need of the substance of interest. The solvents and active ingredients are as discussed with respect to the compositions herein. Thus, the solvent is miscible with water and interstitial fluid, and the at least one active ingredient is soluble in the solvent but insoluble in water and interstitial fluid.

The present application is also directed to methods that include administering to a subject having an allergy or autoimmune condition, an immunotherapy composition provided herein. For example, the immunotherapy composition may include at least one pharmaceutically acceptable active ingredient/substance of interest, selected from the group consisting of modified natural allergens, recombinant and synthetic allergens and their derivatives; and at least one pharmaceutically acceptable non-aqueous solvent. As indicated above, the solvent is miscible with water and interstitial fluid; and the at least one active ingredient is synthesized or modified such that it is insoluble in water and in interstitial fluid, and soluble in the solvent.

As previously indicated, the subject or recipient may be a mammal (as well as other animals), and the mammal may be (but does not have to be) human.

By way of non-limiting example, the administering may include injecting the composition into a muscle of e.g., the mammal, but other forms of administration, such as topical administration (e.g., to the skin of a mammal), intradermal injection, or intraperitoneal injection, are encompassed herein as well. According to non-limiting example embodiments, as discussed above, the present compositions may be administered to a mammal by intraperitoneal injection, although the lymphatic drainage from the intraperitoneal space is different and dilution and precipitation might take place at a different rate resulting in a different size distribution of the resulting particles. Even if particle size distributions are similar, allergen precipitated in different tissues might interact with different populations of antigen-processing or other cells with a different net effect. Intradermal injection may stimulate tumor immunity, e.g., when the composition includes appropriately modified tumor antigens. According to non-limiting example embodiments the present methods may include administering the present compositions by intradermally injecting the composition into a mammal to stimulate tumor immunity, wherein the composition comprises modified, recombinant or synthetic tumor antigens and/or their derivatives.

According to non-limiting example embodiments, examples of the present compositions and methods help individuals having allergies develop a state of immunologic tolerance to the offending allergen(s), such that if the individual is exposed to allergens, the severity, duration, and/or type of reaction may be diminished or completely alleviated as compared to allergic reactions that may have occurred if the individual had not been treated with the present compositions and methods. As is known to those skilled in the art, in other forms of allergen immunotherapy and as also observed with VDBP for poison ivy, a series of increasing doses may both become possible because of developing tolerance and be necessary to achieve optimal levels of tolerance.

Appropriate dosages of the formulations or compositions provided herein may be determined by those skilled in the art, depending on various factors, such as the severity of the allergy (e.g. previous allergic reactions and potential for severe adverse effects if exposed to the allergen) or other ailment being treated, the weight of the subject, the type of allergy or other ailment being treated, etc. Dosage amounts, frequency and total number of doses may be adjusted to achieve desired affects, depending on for example, the subject's/tolerance and reaction to previous doses (if any). A unit dosage may comprise a therapeutically effective amount of e.g., a modified natural, recombinant or synthetic allergen or derivative thereof. A unit dosage will depend upon many factors including age, size, and condition of the individual being treated and the number of times the unit will be taken.

For inhalant allergen immunotherapy sequential doses may be given for example, in clusters with dose increases every 30 minutes for a cluster of usually two or three doses but occasionally, more. Two hours was the originally reported dosing interval when rush immunotherapy was first reported in approximately 1980. Allergists usually do not increase dose at shot intervals greater than 2 weeks during the induction phase of inhalant immunotherapy because of the risk of loss of tolerance. The present inventors conservatively elected to give increasing doses of poison ivy extract at 1-2 week intervals in the immunotherapy of previously untreated patients with poison ivy. Because poison ivy patients are not at risk for the acute severe shot reactions that are possible in inhalant or insect venom immunotherapy, the interval between doses may be much less critical for poison ivy immunotherapy than for immunotherapy to inhalant aeroallergens and stinging insect venoms. For severe food and latex allergy and for many medication allergies there are presently no forms of immunotherapy recognized to be both safe and effective. All of these are potential candidates for the present invention.

Absorption of epinephrine injected for allergic emergencies has been shown to be faster from thigh than from deltoid (upper arm over and just below shoulder). The present inventors used the deltoid muscle for VDBP as it requires less undressing and if the injection site is tender it is generally easier to rest your arm than the leg you walk on. The interstitial fluid water content of both the deltoid and the lateral thigh is sufficiently greater than the 0.15 ml largest single ethanol injection volume the inventors have studied to date that the inventors would not expect injection into these two sites to yield significantly different rates of solvent dilution and resulting particle size distribution. It is possible for some applications, however, that injection into one skeletal muscle or another, or injection or topical application to one area of skin or another, might have an impact on efficacy.

As indicated above, the allergen, may include genetically modified recombinant allergens. Other classes of allergens may be compatible with different methods to achieve the solubility properties needed for this method of vaccine delivery. In particular, recombinant allergens may include allergens manufactured by transferring the gene for the protein to be manufactured into a strain of yeast or bacteria that then produces it in culture. Up to this point there are no known genetically modified recombinant allergens with the solubility properties needed for the vaccine delivery system provided herein, but there is no reason why that could not be done either directly (modifying genes to directly synthesize allergens with the desired solubility properties) or indirectly (modifying genes to synthesize allergens with specific chemical features that would enable or facilitate separate chemical processes to cross-link or otherwise modify them to yield appropriate solubility properties) for the present methods of vaccine delivery.

Example embodiments are also directed to methods of treating an autoimmune disease that include administering to a subject having an autoimmune disease, a composition comprising: at least one pharmaceutically acceptable active ingredient selected from the group consisting of at least one modified natural, recombinant or synthetic tissue or tissue-related allergen and modified allergen or derivative; to induce T-cell immunologic tolerance in the subject (such as a mammal) Administration of such doses may occur anywhere from two hours to two weeks between shots. Corresponding compositions that may be used for the treatment of autoimmune diseases are also encompassed hereby. Each of the ingredients may be as set forth herein with respect to other embodiments.

According to example embodiments, the present invention may apply to liquid as well as non-liquid tissues. When a substance-of-interest is to be administered to a target tissue-of-interest to accomplish a therapeutic purpose, the physical phase (single phase solid, single phase liquid, suspension, emulsion, etc.), particle size (if particulate) and dynamic availability (determined by factors such as solubility and viscosity) as well as mode of administration will all affect the ability of the proposed administration to accomplish its therapeutic purpose. The inventors discovered serendipitously that the administration by injection into a non-liquid tissue with an available water content much greater than the injected volume of solution, of small volumes of a low viscosity solution of a substance-of-interest that is insoluble in water in a pharmaceutically acceptable water-miscible solvent, resulted in the population of a volume of said tissue surrounding the injection site with hundreds of millions of particles in the size range (based on observed effect) of 0.5 to 5 microns in diameter. They observed that with refinement of the method, specifically by increasing the concentration of the active pharmaceutical ingredient in the injected solution, their employment of this combination of parameters resulted in the accomplish of the previously unachieved therapeutic purpose of inducing durable and measurable immunological tolerance to poison ivy in 90% of treated patients.

In the present invention the inventors expand the ability to deliver particles to a therapeutic target by precipitation in three ways:
1. Enable particle delivery by precipitation for substances that do not innately have the properties of insolubility in water combined with solubility in a pharmaceutically acceptable water-miscible solvent by either or a combination of modifying the substance to give it those solubility properties without destroying those of its properties that make it therapeutic and/or by synthesizing a modified version of said substance that either has those solubility properties or is better adapted to modification to confer those solubility properties without loss of the properties that make it therapeutic.
2. Enable delivery to volumes of target tissues that are physically close to physiological barrier membranes including the skin and various mucosal surfaces by topical application in solvents capable of crossing those barrier membranes. (At the time of this application the only such pharmaceutically acceptable solvent is dimethylsulfoxide, DMSO, but it is possible that other solvents may be discovered or developed.)
3. Extend the scope of the invention to liquid as well as non-liquid tissues. The inventors developed the present technology for use in non-liquid tissues with sufficient structure to retain precipitated particles for them to interact with the recipient in the physical forms and locations in which they are formed. Additionally, the inventors envision possible uses in liquid tissues, in this case blood. Two such non-limiting applications in blood are set forth as Examples #4 and #5 herein.
   a. In a non-liquid tissue, the particles that precipitate as the solvent in which they are administered is diluted by the available water content of the tissue will remain in the locations in which they precipitate and interact with the recipient tissue or organism therefrom.
   b. If a solution of the invention is administered to a moving liquid tissue in which the volume of said tissue in which precipitation occurs is significantly diluted by mixing, such as venous blood, the concentration of precipitated particles can rapidly be diluted to the point of insignificance as the volume of blood into which they were administered is mixed with the much larger volume of the same liquid tissue into which the water-miscible solution of the substance-of-interest was not administered.
   c. If a solution of the invention is delivered into a volume of a liquid tissue that is either static or moving slowly in comparison to the speed with which particles within it will settle up or down on the basis of density, particles that are not restrained in place by the structural matrix of a non-liquid tissue will come into contact with each other as they separate from the liquid tissue on the basis of relative density. When they do this, if liquid, they will align themselves in ways that minimize boundary layer energy. If the particles are liquid, they will separate into two separate phases like droplets of oil shaken in water and then allowed to stand. If solid they will agglomerate into clumps, in either case undoing the purpose of the invention without the retraining properties of the structural matrix of a non-liquid tissue.
   d. The present inventors can envision two such uses as non-limiting applications of the method of this invention to blood.

Further example embodiments may include kits and/or systems that include inter alia, one or more of the compositions provided herein. The kits or systems may further include one or more of the following, or other ingredients typically present in composition kits: instructions for use, injection or other administration implements or devices (such as needles, syringes, vials, disinfectant wipes, etc.), disposable implements, additional treatment literature, additional implements or compositions for the treatment of various conditions treated by administration of the present substances of interest, such as e.g., anti-inflammatories, etc.

The following examples are provided to further illustrate various non-limiting embodiments and techniques encompassed by the present invention. It should be understood, however, that these examples are meant to be illustrative and do not limit the scope of the claims. As would be apparent to skilled artisans, many variations and modifications are intended to be encompassed within the spirit and scope of the present disclosure.

### EXAMPLES

### Example 1

### Allergoids of Allergenic Proteins

An allergoid is a derivative of an allergenic protein following modification to reduce its allergenicity (ability to provoke or precipitate an allergic reaction). Allergoids are generally produced by combinations of cross-linking molecules of allergenic proteins with aldehydes of other reactive coupling reagents and changing their physical contour by binding with similar classes of coupling reagents that do not cross-link different molecules. The goal is to disrupt the IgE binding sites at which IgE binding can trigger allergic reactions without disrupting the linear amino acid sequences when presented to antigen-processing cells to progenitors of T-regulatory lymphocytes achieve immunomodulation from sensitization to tolerance. Different coupling reagents and protocols may differ in their ability to produce clinically safe and effective allergoids of different protein allergens.

Traditional allergoid coupling reactions do not confer the solubility properties needed for VDBP. Other solubility-modifying side-chains may be chemically coupled to allergoids to reduce their solubility in water, in many cases to the point of making them water-insoluble for the purpose of VDBP. Most such proteins will be highly soluble in DMSO, however, making them potential candidates for VDBP.

The inventors made and studied allergoids of the major peanut allergen Ara h2. They identified four classes of coupling reagents classes of polymerization reaction for study. These were, cross-linking cysteines with glutaraldelyde+/−formaldehyde &/or other reactive aldehydes, use of succinic acid as an alternate cross-linker of cysteines, carbamylation of lysine residues with potassium cyanate and thiol-esterification of cysteines with monovalent aldehydes such as formaldehyde and valeraldehyde. There are known side-chain coupling reactions to modify protein solubility and we also have novel side chain coupling concepts to further reduce solubility in water and increase it in ethanol. As both polymerization into allergoids and chemical modification of solubility involve coupling to reactive amino acid residues, it is possible that variation in the choice of cross-linking agent, time and conditions of coupling reaction, molar ratio of cross-linking agent to Ara h2 and choice and reaction conditions for solubility-modifying side-chain coupling will differentially affect the tolerogenicity of the resulting materials when used as allergy vaccines.

Proof-of-concept work done includes purification of Ara h2 from commercially available peanut flour, preparation of small quantities of allergoids by reaction with different aldehyde cross-linking reagents, demonstration of differently sized derivatives on molecular sizing gels and demonstration of reduced allergenicity (ability to bind to rabbit anti-Ara h2) of an allergoid made by cross-linking with a proprietary coupling aldehyde reagent.

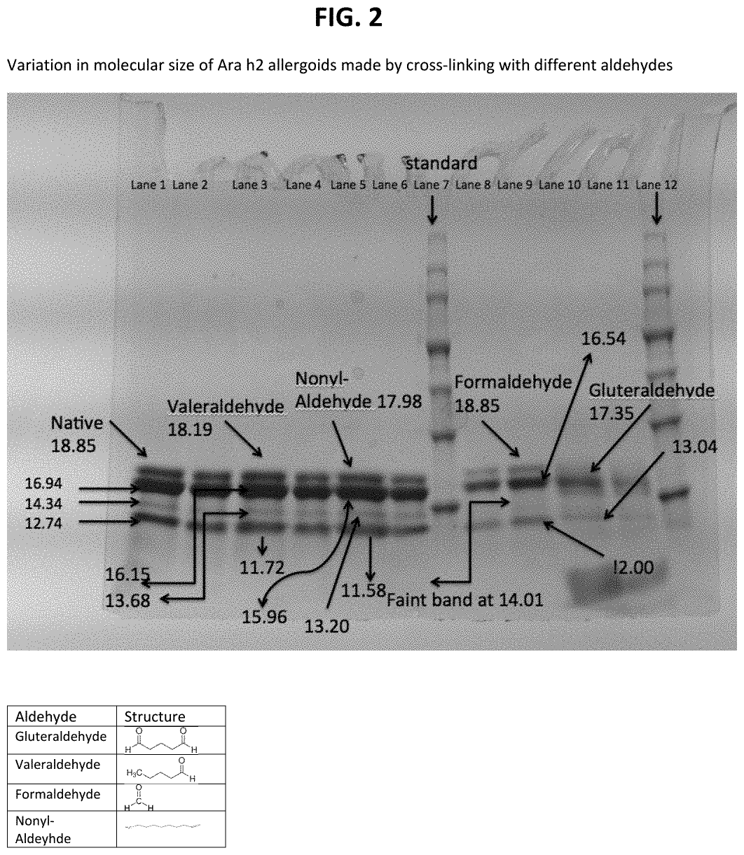

A molecular sizing gel (FIG. 2) shows differences in the size distribution of allergoids produced with different cross-linking reagents.

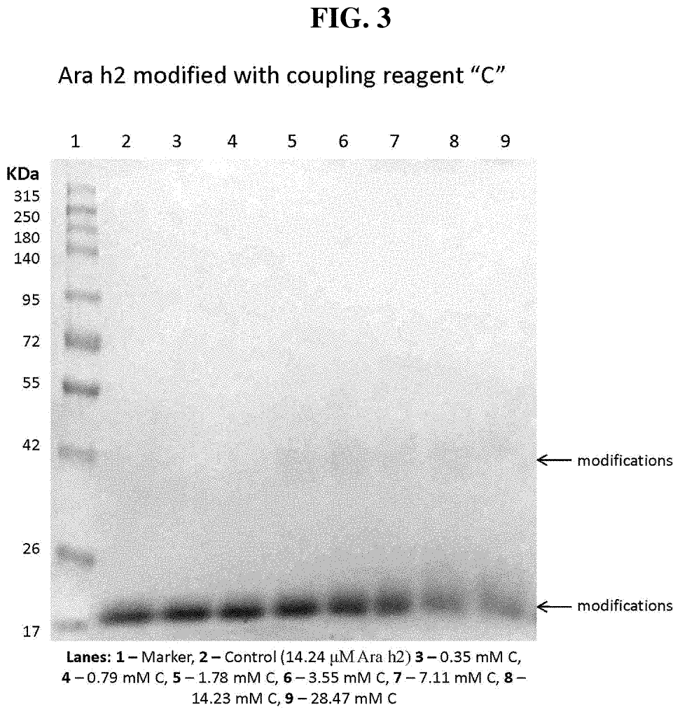

FIG. 3 shows increasing conversion of Ara h2 to allergoid with increasing concentrations of a proprietary aldehyde coupling reagent.

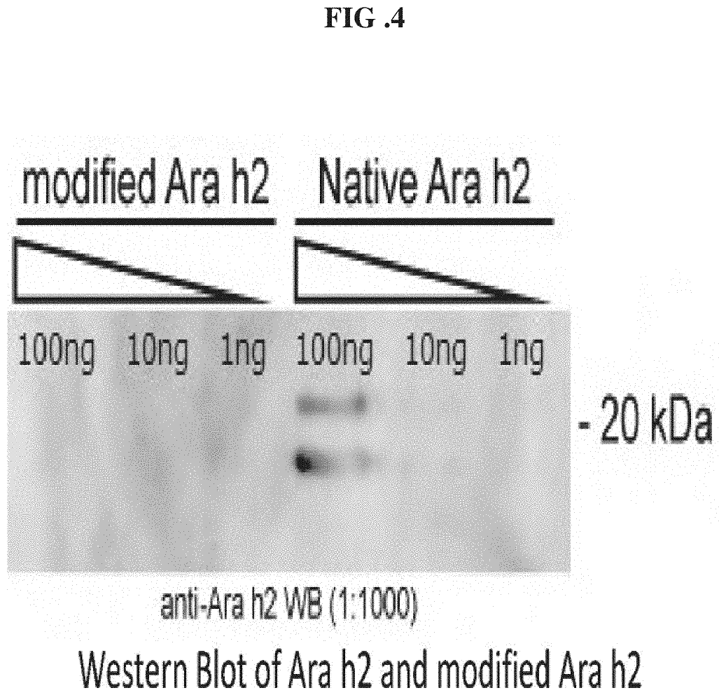

FIG. 4 shows reduced binding of the above allergoid to rabbit anti-Ara h2 by Western Blot.

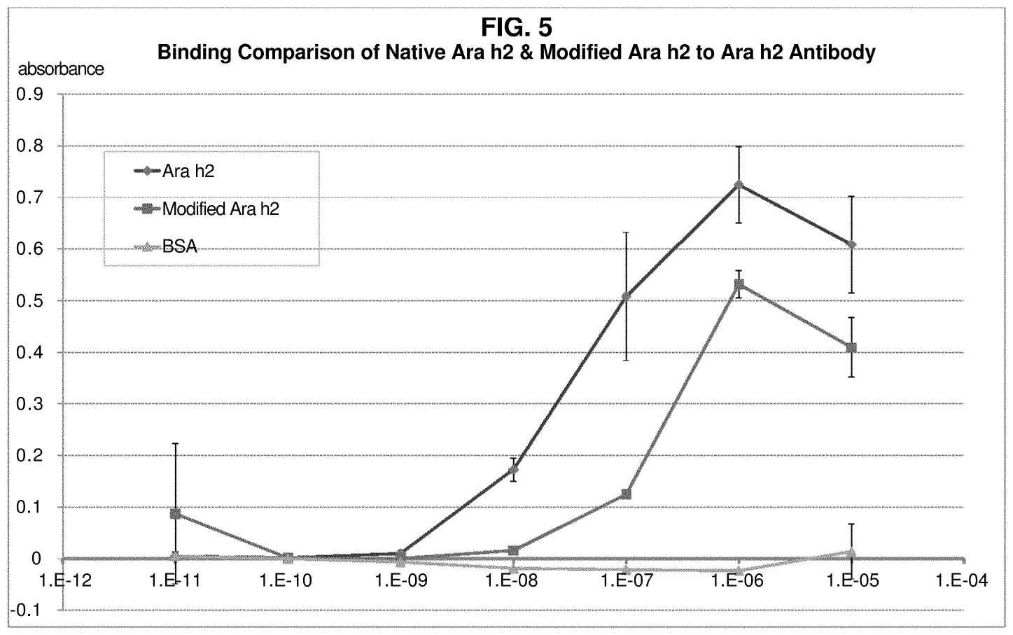

FIG. 5 shows the same reduced binding of the above allergoid to rabbit anti-Ara h2 by ELISA.

This prophetic example considers methods of providing immuno-therapy for peanut allergens, in particular.

The challenge in allergen immunotherapy is to deliver a sufficient quantity of an appropriately configured allergen to cellular microenvironments associated with the development of immunologic tolerance (tolerogenicity), without enough reaching effector signaling microenvironments to trigger allergic reactions (allergenicity). The inventors discovered that in chronic severe poison ivy, immunotherapy with a vaccine formulated to precipitate large numbers of small particles with a large surface area in intimate contact with the cells and cytokines that circulate through muscle, was −200 times more effective at tolerogenicity than the same allergen given by the traditional route, subcutaneous injection in corn oil. Because the process by which tolerogenesis must occur is the same for both peanut allergy and poison ivy (1), the inventors want to make and study the immune response of appropriately formulated peanut allergy vaccines using the same delivery system

A way to formulate a vaccine of Ara h2 (the major peanut allergen) with the necessary solubility properties is to polymerize Ara h2 molecules into allergoids, which constitute a separate and time-honored method of favoring the balance between tolerogenicity and allergenicity (2). Allergoids given by the traditional route of subcutaneous (human) or intraperitoneal (animal) injection in Alum have not been shown to by themselves to induce tolerance in peanut allergy.

Ara h2 allergoids may be produced with the solubility properties needed to synergistically exploit the enhanced tolerogenesis achieved by precipitation of small particles in muscle, and compare their immune system uptake and response to those of the same allergoids administered by traditional methods in a mouse model that could subsequently be used to study response to immunotherapy.

### Significance/Relevance of the Concept:

Anaphylactic IgE-mediated peanut allergy is an increasingly common cause of morbidity, mortality and utilization of health care economic resources with a major but poorly understood genetic component (3) and no safe, simple and effective treatment. The inventors' synergistic application of two methods to enhance T-cell tolerogenesis offers the potential to achieve such a treatment.

### Hypothesis/Concept:

The poison ivy allergen, urushiol, is soluble in 95% ethanol but insoluble in water. Intramuscular injection of urushiol in small volumes of 95% ethanol results in precipitation of large numbers of small insoluble particles as the ethanol is rapidly diluted by interstitial or tissue fluid, resulting in a −200-fold enhancement of tolerogenesis per mg administered urushiol. As the most practical way to make an Ara h2 vaccine with the solubility properties needed to exploit the benefit of this delivery system may be a derivative of a process already known to enhance tolerogenesis, the inventors have the opportunity to see if the synergistic application of two methods of tolerogenesis enhancement yields a sufficiently high ratio of tolerogenicity to allergenicity to constitute a predictably safe and effective treatment for peanut allergy.

### Objectives:

1) Synthesize allergoids of Ara h2 that are insoluble in water but highly soluble in 95% ethanol or other water-miscible solvents of which small volumes can be safely injected into muscle. 2) Identify particle size and stability following injection into muscle in a mouse model. 3) Determine whether injected allergoid remains in situ or is taken up by dendritic cells, as this would define parameters to be measured in subsequent studies of immunotherapy efficacy.

### Methods:

Synthesis: Standard cross-linking of Ara h2 with glutaraldehyde &/or formaldehyde. Evaluation of products of differing molecular size and cross-link density for appropriate solubility. Addition of hydrophilic or hydrophobic sidechains by standard methods of protein chemistry as needed to optimize solubility profile.

### Analysis:

Comparison of mouse immune response to fluorescein labeled and unlabeled allergoid administered by traditional (intraperitoneal in alum) and experimental (intramuscular in ethanol or alternate solvent) routes. Compare uptake by circulating antigen-presenting cells by fluorescence-activated cell sorting and study persistence of allergen in situ and migration into regional lymphoid tissue.

The strategies of interest involve one or more of modifying an allergen, modifying the way it is delivered, and modifying the response of various host receptor and antigen-processing tissues. The common goal is to increase the efficiency with which allergen is delivered to the cells and microenvironments associated with the induction of tolerance and reduce its exposure to the cells and microenvironments that favor the triggering of allergic reactions. The present inventors propose to combine two different methods of this type. One, the polymerization of allergen molecules by cross-linking to form allergoids, has been known for decades but never found by itself to provide sufficient control of allergen trafficking for safe peanut allergy immunotherapy. The other, formulation of a vaccine with solubility properties that precipitate large numbers of small particles of allergen with a large total surface area in intimate contact with the cells and cytokines that circulate through muscle tissue, is novel and has not previously been applied to diseases of humoral immunity. The inventors believe that a synergistic combination of the two methods has the potential to achieve sufficient control of allergen trafficking to permit safe, effective and reliable immunotherapy for peanut allergy.

The scope of work to be performed with this includes the production of allergoids of major peanut allergen Ara h2 that are insoluble in water and interstitial or tissue fluid but highly soluble in either 95% ethanol or in another solvent that mixes freely with water and of which small volumes can safely be injected into muscle. The inventors will then inject these vaccines to the thigh muscle of mice and track their clearance by quantitative immune analysis of the serum and the local muscle tissue. It is hypothesized that precipitation into large numbers of small particles occurs as the ethanol or other solvent is rapidly diluted by interstitial or tissue fluid following injection. Fluorescein labeling of the allergoid and Fluorescence Activated Cell Sorting (FACS) analysis will be used to assess uptake by antigen presenting cells and to follow the persistence of allergen in situ, and of the labeled antigen-processing cells migration into regional lymphoid tissue. These studies may shed light on which of two possible mechanisms of tolerogenesis predominates: long term persistence in situ until the injected allergen is tolerated like self, or uptake by dendritic cells with further processing in regional lymph nodes. Allergenicity of the Ara h2 and the allergoid preparations delivered into either conventional sites (intraperitoneally) or intramuscularly, will be compared by assessing the cytokine and immunoglobulin profile of the mice.

It is believed that successful completion of this project will lead to both trials of immunotherapy in a mouse model of peanut allergy and further mechanistic studies to investigate the immune processes that operate during induction of tolerance to allergen exposure.

### DISCUSSION

### Background

The same mechanism of T-regulatory cell tolerogenesis is common to humoral and cell mediated immunity. A vaccine delivery system that successfully induced tolerance to poison ivy may be similarly effective in humoral allergy.

Conversion of protein allergens into allergoids by cross-linking or other methods increases their ratio of tolerogenicity to allergenicity, presumably by reducing access by cell-bound IgE molecules to epitopes able to trigger cross-linking Allergoids act to expand the tolerogenic T-regulatory cell population in competition with Th1 and Th2 enhancement of allergic sensitization. As the inventors' vaccine delivery system appears to enhance T-cell population shift in the same pro-tolerogenic direction it is possible that the two interventions in combination may be synergistic, i.e., more effective than simply additive.

The inventors propose to make allergoids of Ara h2 that are soluble in ethanol but insoluble in water, and study the same vaccine delivery system in a mouse model of peanut allergy.

### Methods:

The inventors propose to make allergoids by four methods: 1) cross-linking cysteines with glutaraldelyde 2) cross-linking cysteines with succinic acid; 3) carbamylation of lysine residues with potassium cyanate; and 4) thiol-esterification of cysteines with monovalent aldehydes such as formaldehyde and valeraldehyde.

There are known side-chain coupling reactions to modify protein solubility and the inventors have designed novel side chain coupling concepts to further reduce solubility in water and increase it in ethanol. As all of these reactions involve coupling to reactive amino acid residues, it is possible that variation in the choice of cross-linking agent, time and conditions of coupling reaction, molar ratio of cross-linking agent to Ara h2 and choice and reaction conditions for solubility-modifying side-chain coupling will differentially affect the tolerogenicity of the resulting materials when used as allergy vaccines. The inventors will therefore make differently formulated allergoids that form low viscosity solutions in ethanol and are insoluble in water.

### Preliminary Results:

The inventors made small batches of glutallergoid varying the glutaraldehyde (glut): Ara h3 molar ratio from 4 to 10 and valerallergoids over a 3-fold range of valeraldehyde: Ara h2. A molecular sizing gel is shown in FIG. 5. The glutarallergoids formed at higher glut: Ara h2 ratios show reduced solubility in water. Valerallergoids have not yet been purified to study their solubility.

### Follow up:

The inventors plan to increase both cross-linking of and hydrophobic binding to Ara h2 until allergoids that are insoluble in water are obtained, and then make them soluble in ethanol by N-glycosylation. Tolerogenesis will be studied with allergoids that have the necessary solubility properties in mouse and possibly also rat models of peanut allergy

### Example 2: Peptide Vaccines

Peptide allergy vaccines typically comprise sets of overlapping peptides, usually 10 amino acids in length, representing the amino acid sequence of epitopes of allergenic proteins associated with allergic reactivity. Several such vaccines looked promising in preclinical trials in animal models and some also in Phase 1 clinical trials but failed to meet target endpoints in Pivotal Clinical Trials.

Considerable innovation has gone into the formulation of peptide vaccines but much less into the choice of routes by which they can be delivered. The most commonly used route of administration is subcutaneous injection of solutions of the mixed peptides in phosphate-buffered saline with 25% glycerine being the most common. This is not a particularly effective way to feed antigen to antigen-processing cells.

There are methods to couple the same solubility-modifying “tails” to both the C-terminal and N-terminal ends of most peptides without significant binding to C-terminal or N-terminal sidechains of the amino acids that comprise those peptides. With appropriate selection of coupling reagents these mixes of overlapping peptides can be rendered almost uniformly water-insoluble. Almost all of these products will be soluble in DMSO, and many can be made soluble in ethanol, making them appropriate candidates for VDBP. Injection of DMSO solutions of these modified peptide vaccines into skeletal muscle should result in much more efficient uptake by the tolerizing lineages of antigen-processing cells that populate skeletal muscle.

The peptides that comprise complete overlapping sets vary widely in solubility and when the route of administration to be studied is subcutaneous injection (i. e., without the present invention), those peptides that are insoluble in water must be left out.

While one skilled in the art may make his or her own allergoids, as done in the exploration of Example 1 of this invention, it is a more difficult and expensive task to make one's own peptide vaccine.

### Example 3: Nucleic Acid Vaccines

Nucleic acid vaccines consist of DNA or RNA that codes proteins to which one wants to modulate the immune response. They were historically developed as a way to immunize against pathogenic viruses stripped of the components that make them infectious. There is no reason why the same technology can't be applied to the nucleic acid sequences that code for allergenic proteins. Like peptide vaccines in which the peptide chains are too short to bind with the binding sites of IgE molecules, nucleic acid vaccines are incapable of provoking IgE mediated allergic reactions and thus safe to use for the treatment of IgE-mediated allergies.

Nucleic acid vaccines are currently administered by coupling to viral vectors, benign viruses that infect cells throughout the body and carry their contents into the cells they infect, where they hopefully infect enough lymphocytes of the proper classes to induce protective sensitization.

Nucleic acid vaccines can be rendered insoluble in water by coupling at their ends to the same classes of solubility-modifying reagents discussed for both allergoids and peptide vaccines. Both coupled and not-coupled nucleic acid vaccines will be soluble in DMSO and many can be made soluble in ethanol. These solubility-modified substances-of-interest can then be administered via VDBP, to skeletal muscle when the objective is immunomodulation from sensitization to tolerance and to the dermis when the objective is immunomodulation from tolerance to sensitization.

The inventors expect that feeding nucleic acid vaccines to antigen-processing cells in the form of 0.5 to 5 micron particles and selectively doing so in tissues in which those cells are of predominantly tolerizing or sensitizing lineages will be a more effective stimulus to therapeutic immunomodulation than the present (prior art) alternative of injecting them coupled to viral vectors that are much less selective in their choice of targets.

As is the case with peptide vaccines, implementation of this example requires collaboration with an owner or producer of nucleic acid vaccines and is beyond the capabilities of the inventors by themselves.

### Examples Involving a Liquid Target Tissue (Blood)

### Example 4

The present application is further intended to encompass any other therapeutic agent or combination of agents for which the present route of delivery to the body enhances effectiveness, reduces adverse effects, or both. An example involving the liquid tissue of the circulatory system could be an effective way to deliver a locally acting drug to unidentified microscopic metastases following removal of cancers that carry a risk of micrometastatic spread. A method could be to polymerize a slowly activated metabolic precursor of the drug and couple it to a genetically engineered high affinity antibody to a tumor-specific antigen or antigens of that cancer. If given by slow intravenous injection the drug would bind to tumor cells wherever they might be where the activity of the drug would persist. The antibody-attached drug would not persist except where attached to tumor cells by antibody binding, minimizing toxicity to other tissues. Such methods, compositions and uses are intended to be encompassed hereby.

### Example 5

This non-limiting example would be application of the method of this invention to embolize the vascular supply of tumors nourished by arteries that are accessible by radiologically guided intra-arterial catheter, possibly with substance that have pharmaceutical activity as well as the ability to obstruct small blood vessels. Solutions of candidate substances in small volumes of pharmaceutically acceptable solvents could be injected into arteries serving such tumors so that the precipitated particles would obstruct the smaller branches of the arterial tree that serve the target tumor.

The particle sizes needed for arterial, arteriolar or capillary occlusion would be larger than the 0.5 to 5 micron diameter for effective uptake by antigen-processing cells. These larger sizes could be achieved, for example, by increasing the viscosity of the injected solution

Although the present disclosure has been described in example embodiments, additional modifications and variations would be apparent to those skilled in the art. It is therefore to be understood that the present disclosure herein may be practiced other than as specifically described. Thus, the present embodiments should be considered in all respects as illustrative and not restrictive. Accordingly, it is intended that such changes and modifications fall within the scope of the present disclosure as defined by the claims appended hereto.

What is claimed is:

1. A method of populating a target tissue of interest of a subject, in which tissue it would be safe to precipitate particles of water-insoluble therapeutic agents in situ, with precipitated particles of a modified or synthesized substance of interest, in which tissue the precipitated particles remain where they are formed for a sufficient time to begin interaction with physiology of the subject in a physical state and locations in which they were formed, in the target tissue where formed, comprising
precipitating within said target tissue said particles of the modified or synthesized substance of interest, wherein said modified or synthesized substance of interest does not, as it exists in nature in its unmodified and unsynthesized form, have properties of being insoluble in water but sufficiently soluble in a pharmaceutically acceptable, water-miscible non-aqueous solvent such that doses can be administered to target tissues in small enough volumes of said pharmaceutically acceptable, water-miscible non-aqueous solvent to be diluted rapidly enough by the water content of the target tissue of interest to precipitate in particles spanning an effective size range to achieve a desired physiological or pharmacological effect,
wherein said modified or synthesized substance of interest is either a modified substance, which is modified from a substance's original form to impart solubility properties not present in an original unmodified form of the substance, or a synthesized substance that is synthesized in such a way to confer solubility properties;
wherein said precipitating takes place upon administering a solution of the modified or synthesized substance of interest to the subject; said solution comprising a pharmaceutically acceptable, water-miscible non-aqueous solvent, and the modified or synthesized substance of interest that is soluble in the pharmaceutically acceptable, water-miscible non-aqueous solvent, and that is insoluble in tissue fluids of the target tissue of interest; wherein the pharmaceutically acceptable, water-miscible non-aqueous solvent is freely miscible with the tissue fluids of the target tissue of interest, and
wherein the solution is administered to the tissue of the subject in a volume of the pharmaceutically acceptable, water-miscible non-aqueous solvent such that the solution will be diluted by tissue fluids in the target tissue of interest following administration, at a rate that results in precipitation of an effective mass of the modified or synthesized substance of interest with a distribution of particle sizes that will accomplish said desired physiological or pharmacological effect for which the modified or synthesized substance of interest is administered.

2. The method of claim 1, wherein the solution is administered to the target tissue by injection.

3. The method of claim 2, wherein the pharmaceutically acceptable, water-miscible non-aqueous solvent comprises ethanol and the injection is intramuscular injection.

4. The method of claim 1, wherein the pharmaceutically acceptable, water-miscible non-aqueous solvent is capable of diffusing across phospholipid membranes and the solution is administered by topical application.

5. The method of claim 4, wherein the pharmaceutically acceptable, water-miscible non-aqueous solvent comprises DMSO.

6. The method of claim 1, wherein the tissue is a non-liquid tissue.

7. The method of claim 6, wherein the target tissue comprises a tissue selected from the group consisting of muscle, skin, and tissues underlying mucus membranes of the subject.

8. The method of claim 1, wherein the solution is a homogenous solution.

9. The method of claim 1, wherein the modified or synthesized substance of interest comprises modified allergens, modified and natural synthetic allergens, modified tumor antigens, and modified or synthesized natural or recombinant autologous antigens and their derivatives.

10. A method of populating a target tissue of interest of a subject, with precipitated particles of a modified or synthesized substance of interest, which precipitated particles remain in the target tissue where formed, comprising
administering a solution to a target tissue of interest of a subject, said solution comprising:
a pharmaceutically acceptable, water-miscible non-aqueous solvent, and
the modified or synthesized substance of interest, wherein the modified or synthesized substance of interest is soluble in the pharmaceutically acceptable, water-miscible non-aqueous solvent and insoluble in tissue fluids of the target tissue of interest; and wherein the pharmaceutically acceptable, water-miscible non-aqueous solvent is freely miscible with the tissue fluids of the target tissue of interest;
wherein the solution is administered to the target tissue of interest of the subject in an effective volume of the pharmaceutically acceptable, water-miscible non-aqueous solvent, such that the pharmaceutically acceptable, water-miscible non-aqueous solvent will be diluted by tissue fluids in the target tissue of interest following administration to the tissue, at a rate that results in precipitation of an effective dose of particles of the modified or synthesized substance of interest in the target tissue of interest with a distribution of particle sizes of the substance of interest that will accomplish a purpose for which the modified or synthesized substance of interest is administered;
wherein said modified or synthesized substance of interest is either a modified substance, which is modified from a substance's original form to impart solubility properties not present in an original unmodified form of the substance, or a synthesized substance that is synthesized in such a way to confer solubility properties; and
wherein said modified or synthesized substance of interest does not, as it exists in nature in its unmodified and unsynthesized form, have properties of being insoluble in water but sufficiently soluble in a pharmaceutically acceptable, water-miscible non-aqueous solvent such that doses can be administered to target tissues in small enough volumes of said pharmaceutically acceptable, water-miscible non-aqueous solvent to be diluted rapidly enough by the water content of the target tissue of interest to precipitate in particles spanning an effective size range to achieve a desired physiological or pharmacological effect.

11. The method of claim 10, wherein the solution is administered to the target tissue by injection.

12. The method of claim 11, wherein the pharmaceutically acceptable, water-miscible non-aqueous solvent comprises ethanol and the injection is intramuscular injection.

13. The method of claim 10, wherein the pharmaceutically acceptable, water-miscible non-aqueous solvent is capable of diffusing across phospholipid membranes and the solution is administered by topical application to the subject.

14. The method of claim 11, wherein the pharmaceutically acceptable, water-miscible non-aqueous solvent comprises DMSO.

15. The method of claim 10, wherein the tissue is a non-liquid tissue.

16. The method of claim 15, wherein the target tissue comprises a tissue selected from the group consisting of muscle, skin, and tissues underlying mucus membranes of the subject.

17. The method of claim 10, wherein the solution is a homogenous solution.

18. The method of claim 10, wherein the modified or synthesized substance of interest comprises modified allergens, modified and natural synthetic allergens, modified tumor antigens, and modified or synthesized natural or recombinant autologous antigens and their derivatives.
